SEQUENCE LISTING (15 sequences)

SEQ ID NO: 1 - DNA, 2127 bp, Artificial Sequence
  The sequence is a synthesized SV40LT whole gene sequence
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcaatacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaactaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagaggataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaactggagttttagattggctaagaaacagtgataatgatgatgaagacagccaggaaaatgctgataaaaatgaagatggtggggagaagaacatggaagactcagggcatgaaacaggcattgattcacagtctcaaggctcatttcaggcccctcagccctcacagtcctcacagtctgttcatgatcagccatatcacatttgtagaggttttacttgctttaaaaaacctcccacacctccccctgaacctgaaaca

SEQ ID NO: 2 - DNA, 24 bp, Artificial Sequence
  The sequence is synthesized.
ggatgcttcctctagtctgtgatg

SEQ ID NO: 3 - DNA, 25 bp, Artificial Sequence
  The sequence is synthesized.
ctctagcctacccagaactgcagag

SEQ ID NO: 4 - DNA, 20 bp, Artificial Sequence
  The sequence is synthesized.
tcagaaggtgccaacctcaa

SEQ ID NO: 5 - DNA, 20 bp, Artificial Sequence
  The sequence is synthesized.
cgtagcttgacctgcgagtg

SEQ ID NO: 6 - DNA, 20 bp, Artificial Sequence
  The sequence is synthesized.
ccatgcgagaggagaatggt

SEQ ID NO: 7 - DNA, 20 bp, Artificial Sequence
  The sequence is synthesized.
tgcggatgagagcttctgtc

SEQ ID NO: 8 - DNA, 20 bp, Artificial Sequence
  The sequence is synthesized.
ccgaggcagaatggagtgta

SEQ ID NO: 9 - DNA, 20 bp, Artificial Sequence
  The sequence is synthesized.
cagtggcaacagcaagatca

SEQ ID NO: 10 - DNA, 21 bp, Artificial Sequence
  The sequence is synthesized.
ggccaagacctctgccttatt

SEQ ID NO: 11 - DNA, 20 bp, Artificial Sequence
  The sequence is synthesized.
gtcggagacagcaatgttcg

SEQ ID NO: 12 - DNA, 20 bp, Artificial Sequence
  The sequence is synthesized.
attgcctggaacgcagtgag

SEQ ID NO: 13 - DNA, 24 bp, Artificial Sequence
  The sequence is synthesized.
cctgagtcttccatgttcttctcc

SEQ ID NO: 14 - DNA, 21 bp, Artificial Sequence
  The sequence is synthesized.
gcctcttgtggatgagcctaa

SEQ ID NO: 15 - DNA, 20 bp, Artificial Sequence
  The sequence is synthesized.
ccaaggactctgtgcagcat

(19) United States
(12) Patent Application Publication
(10) Pub. No.: US 2019/0345441 A1
(43) Pub. Date: Nov. 14, 2019

(54) Preparation and application of immortalized alpha-1,3-galactosyltransferase gene knockout pig hepatocyte cell line

(71) Applicants: Kefeng DOU, Xi'an (CN); Xiao LI, Xi'an (CN)

(72) Inventors: Quancheng Wang, Xi'an (CN); Kefeng Dou, Xi'an (CN); Xiao Li, Xi'an (CN); Xuan Zhang, Wuhan (CN); Bo Wang, Xi'an (CN); Ge Bai, Xi'an (CN)

(73) Assignees: Kefeng DOU, Xi'an (CN); Xiao LI, Xi'an (CN)

(21) Appl. No.: 16/503,481

(22) Filed: Jul. 4, 2019

(30) Foreign Application Priority Data
May 4, 2018 (CN) 201810417149.8

(51) Int. Cl. C12N 5/071; G01N 33/50

(52) U.S. Cl. CPC C12N 5/067; G01N 33/5067; C12N 2510/04; C12N 2320/10; G01N 2500/04; C12N 2503/04; G01N 2500/10; G01N 2500/02; C12N 2503/02

(57) ABSTRACT

An immortalized α-1,3-galactosyltransferase gene knockout (GTKO) pig hepatocyte cell line, the preparation method and its application in preparing bioartificial liver and preparing medicine for treating liver failure. The immortalized GTKO pig hepatocyte cell line of this invention retains the main characteristics of the primary GTKO pig hepatocytes, including the function of urea synthesis and albumin synthesis. The cell line can be subjected to near-unlimited expansion culture in vitro, and can be used to study key molecular and drug intervention targets in xenograft rejection. When applied to bioartificial liver treatment, the immortalized GTKO pig hepatocytes can effectively solve the problem of xenogeneic hyperacute immune rejection, and reducing the use of immunosuppressive agents, prolonging the survival of transplant recipients and the time of normal liver function. Thus, the immortalized GTKO pig hepatocytes have important medical application prospects.

### CROSS REFERENCE TO THE RELATED APPLICATIONS

This application is based upon and claims priority to Chinese Patent Application No. 201810417149.8, filed on May 4, 2018, the entire contents of which are incorporated herein by reference.

### TECHNICAL FIELD

This invention relates to the field of organ transplantation medical biotechnology. Specifically, it relates to a method for preparing a pig hepatocyte cell line for transplantation, particularly an immortalized pig liver cell line.

### BACKGROUND

Liver Transplantation is the Most Effective Treatment for End-stage Hepatic Disease

Lots of patients have lost their lives due to the delayed treatments because of the shortage of liver donors. The key component of bio-artificial liver is the bioreactor which is made of bioactive cells and its supporting system providing cell growth and metabolism microenvironment. The cells' properties and functions of bioreactors are the main factors that determine the efficiency of bio-artificial liver. Bioreactors use fresh piglet hepatic cells as its bioactive materials for few reasons: first, piglet hepatic cells have content of cytochrome P450 and mixed oxidase activity similar to that of human liver cells. Pig hepatic cells have better functionality of Bilirubin metabolism and blood ammonia clearance. Besides, they are very cheap and easy to obtain. Therefore, piglet hepatic cells are used for bioactive cells in bio-artificial liver.

The existing technology of xenotransplantation uses pig livers as the liver source which is a good solution for donor shortage problem. However, the use of pig hepatic cells to prepare bio-artificial livers leads to the occurrence of immune rejection due to the cross-species barriers, especially the occurrence of hyper-acute immune rejection in xenotransplantation, which is still the main factor restricting the normal function of grafts and the long-term survival of transplant recipients.

The record for the longest survival time of xenotransplantation is 29 days. Researches have proved that acute immune rejection occurred after the xenotransplantation and, at present, the main method to prevent the rejection is the use of immuno-suppressants targeting immune cells. We can interfere the important drug target sites by studying the key molecules of hepatic parenchymal cells mediating immune rejection in xenograft, which can reduce the use of immunosuppressive agents, and extend the survival time of transplant recipients and the normal function time of transplanted liver.

Using α-1,3-galactosyltransferase (αGT) gene knockout pig hepatic cells as the xenograft donor liver cells is an important solution to study the mechanism of immune rejection of xenotransplantation. The following problems still exist in the use of primary hepatocytes: 1. The extraction of primary hepatocytes from GTKO pigs is complicated, and it requires relatively high operating techniques to obtain highly vigorous hepatocytes; 2. The primary hepatocytes of GTKO pig have a short culture time in vitro, and the longest culture time is on week. The primary liver cells do not proliferate in vitro, and they would gradually undergo apoptosis and necrosis due to the separation from the environment in vivo. 3. Primary hepatocytes cultured in vitro are hard to conduct gene interference, and the results of scientific research are poor in repeatability. Therefore, the short survival time of liver transplantation is an important bottleneck which limits the hepatic xenotransplantation.

In the field of hepatocyte transplantation of xenotransplantation, it is urgent to solve the problems of hyper-acute immune rejection in xenografts and the primary hepatocytes defects in operation.

The immortalization of hepatic cells is expected to be a key to the short survival time of liver transplantation. SV40, short for simian virus 40, is a tumorigenic virus found in both humans and monkeys, which consists of three structural proteins, VP1, VP2, and VP3, and two antigens, LT and ST. In recent years, researchers have found that SV40LT antigen gene can make cell proliferation and immortalization. SV40LT induced immortalized cells have been widely used in vitro experiments to illustrate the mechanisms of the limited life cycles, aging and immortalization.

However, there are few studies on the immortalization of liver cells in GTKO pigs in the field of xenotransplantation. The study on the cell lines of GTKO pigs liver cells is a great and significant progress of liver transplantation technology and organ transplantation in medical science.

### SUMMARY

The cultivation of α-1,3-galactosyltransferase (αGT) knockout pigs (GTKO pigs) effectively solved the occurrence of xenogeneic hyperacute immune rejection, while acute rejection remains the main factors limiting the long-term survival of transplant recipients and the normal function of the graft. This invention provides solution for the short survival time caused by acute rejection of the xenogeneic liver transplantation and the increased risk of receptor infection due to the extensive use of immunosuppressive agents.

The highly viable primary hepatocytes were isolated from GTKO pigs. The immortalized GTKO pig hepatocyte cell line was established by infecting SV40LT lentivirus, and its functions and characteristics were identified. The cell line can provided a research basis for solving the acute rejection of xenogeneic liver transplantation.

In one aspect, this invention provides an immortalized α-1,3-galactosyltransferase (αGT) gene knockout pig hepatocyte cell line HepDT. The immortalized GTKO pig hepatocyte cell line HepDT was deposited on May 25, 2018 in the China General Microbiology Culture Center Collection Center (CGMCC) with the accession number CGMCC No. 15590. The deposit address is Institute of Microbiology, Chinese Academy of Sciences, Datun Road, Chaoyang District, Beijing, Postcode 100101

The immortalized GTKO pig hepatocyte cell line HepDT was obtained by transfecting lentiviral vector containing SV40 T antigen gene and human telomerase catalytic subunit gene into the extracted primary GTKO porcine normal hepatocytes. At last, HepDT was obtained by cloning screening.

In a second aspect, the invention provides a method for preparing an immortalized α-1,3-galactosyltransferase gene knockout (GTKO) pig hepatocyte of the first aspect, comprising the following steps:
1. GTKO pig primary hepatocytes extraction.
2. SV40LT recombinant lentivirus construction.
3. Primary GTKO pig hepatocytes infected with recombinant lentiviruses and screened for monoclonal cells.
4. The identification of immortalized GTKO pig hepatocytes HepDT.

In some embodiments, the alpha-1,3-galactosyltransferase knockout pig primary hepatocytes of step 1 were extracted using a modified classical Seglen II collagenase/DMEM two-step method in combination with a stable peristaltic pump perfusion system.

The modified classical Seglen II collagenase/DMEM two-step method for primary hepatocyte extraction includes the following steps:
(1) GTKO pigs born about 20 days old are selected and pre-infusion preparation includes hunger and anesthesia.
(2) Aseptically incision of the abdominal cavity, preliminary separation of the hepatic portal vein and the inferior vena cava. Then catheterization, sterile line ligation.
(3) Infusion of saline from the inferior vena cava.
(4) GTKO pig liver was anatomized and removed, then perfused with II collagenase/DMEM through a peristaltic pump system.
(5) The digestion reaction was terminated with complete medium. The hepatocytes obtained from the liver tissue were washed, filtered, centrifuged, and the precipitate was suspended and lysed with red blood cell lysate.
(6) Suspension of the obtained cells using red blood cell lysate and centrifugation.
(7) Following that, the primary cells were suspended and incubated in a culture flask until they are adherent, then changing the medium, removing unattached cells, and continue to culture.

In some embodiments, the construction of the SV40LT gene or human telomerase catalytic subunit (hTERT) gene recombinant lentivirus comprises the steps of:
(1) The SV40LT whole gene sequence (SEQ ID NO: 1) or the htert gene sequence was synthesized and ligated into the pHBLV-CMV vector to obtain the recombinant vector pHBLV-CMV SV40LT carrying the SV40LT sequence and the recombinant vector pWPT-hTERT carrying the hTERT sequence.
(2) The SV40LT overexpressed recombinant vector pHBLV-CM SV40LT was purified and amplified using DH5a E. coli.
(3) The plasmid vectors pSPAX2, pMD2G and SV40LT were amplified and extracted by high purity endotoxin-free kit, and transfected into 293T cells together. The cells culture medium was changed to complete medium 6 hours after transfection.
(4) After 293T cells were cultured for 48 h and 72 h, the supernatants containing the lentiviral particles were collected and centrifuged to remove the supernatant of the cell debris.
(5) The supernatant was ultracentrifuged to obtain a high titer of the SV40LT gene recombinant lentivirus for the construction of immortalized GTKO pig hepatocyte cell line.

In some embodiments, the construction of human telomerase catalytic subunit (hTERT) gene recombinant lentivirus for GTKO pig hepatocytes comprises the following steps:
(1) The hTERT gene sequence was synthesized and ligated into the pWPT vector to obtain the recombinant vector pWPT-hTERT carrying the hTERT gene.
(2) The overexpressed plasmid pWPT-hTERT was amplified by DH5a Escherichia coli, then the overexpression plasmid was extracted and purified.
(3) The plasmid vectors pSPAX2, pMD2G and hTERT were amplified and extracted by high purity endotoxin-free kit, and transfected into 293T cells together. The cells culture medium was changed to complete medium 6 hours after transfection.
(4) After 293T cells were cultured for 48 h and 72 h, the supernatants containing the lentiviral particles were collected and centrifuged to remove the supernatant of the cell debris.
(5) The supernatant was ultracentrifuged to obtain a high titer of the hTERT gene recombinant lentivirus for the construction of immortalized GTKO pig hepatocyte cell line.

In some embodiments, the immortalized GTKO pig hepatocyte cell line was prepared by transfecting SV40LT antigen gene and human TERT gene recombinant lentiviral vector into the extracted primary porcine hepatocyte. The immortalized GTKO pig hepatocyte cell line was obtained by clonal screening. The lentiviral vectors pHBLV-CMV SV40LT and pWPT-hTERT were transfected into GTKO pig hepatocytes by the following methods: The lentiviral vectors pWPT-SV40Tag and pWPT-hTERT containing the SV40 T antigen or human telomerase catalytic subunit gene were constructed separately. Lentiviral particles with SV40 LT antigen or htert gene were packaged as infectious but replication defective. At last the lentiviral particles were used to infect GTKO pig primary hepatocytes.

In some embodiments, the SV40LT lentivirus, hTERT lentivirus is purchased from Hanheng Biotechnology (Shanghai) Co., Ltd. In some embodiments, the SV40LT lentivirus infected GTKO pig normal pig hepatocytes and screening the monoclonal cells as follows:
(1) The SV40LT recombinant lentivirus is used to infect GTKO pig primary liver cells.
(2) Continue to culture with normal medium.
(3) Screening of successfully lentivirus infected GTKO pig hepatocytes using complete medium containing antibiotics.
(4) Survived recombinant GTKO pig hepatocyte single cell continued to subculture in the cell plate.
(5) After subculturing, the cells were transferred to a culture flask and continued to culture to obtain the immortalized GTKO pig hepatocyte cell line.

This invention used a recombinant retrovirus containing a recombinant plasmid to introduce the SV40 large T antigen gene or hTERT gene into the primary GTKO pig hepatocytes, and established an immortalized GTKO pig liver cell line.

The basic principle of lentiviral particles with SV40 T antigen or hTERT gene with infectious ability but replication defects is that the lentiviral vector system consists of two parts, a packaging component and a carrier component.

A lentiviral vector refers to a viral vector derived from human immunodeficiency virus-1 (HIV-1). The lentiviral vector contains the genetic information required for packaging, transfection, and stable integration, which is a major component of the lentiviral vector system. The lentiviral vector carrying the foreign gene is packaged into an infectious virus particle with the help of the lentiviral packaging plasmid and the 293T cell line. Then the foreign gene is expressed in the cell or living tissue by lentiviral infecting the cell or the living tissue.

In this application, the packaging component of the lentiviral vector used to infect primary hepatocytes consist of HIV-1 genome without cis-acting sequences required for packaging, reverse transcription and integration, which can provide the proteins necessary to produce viral particles in trans. The lentiviral packaging components are usually constructed separately into two plasmids, one expressing the Gag and Pol proteins and the other expressing the Env protein. The purpose is to reduce the possibility of recombinant lentivirus recovery to the wild type virus.

By co-transfecting three plasmids of the viral packaging component and the carrier component into cells (such as human 293T cells), virus particles carrying the gene of interest can be harvested in the cell supernatant with only one-time infection ability and no replication ability.

The vector component is complementary to the packaging component, ie, contains the HIV cis-acting sequence required for packaging, reverse transcription and integration, and has a multiple cloning site of the target gene inserted under the control of the heterologous promoter and the target gene of insertion at this site.

In order to reduce the possibility that the homologous recombinant virus of two components restored to the wild-type virus. It is necessary to minimize the homology between the two components, such as replacing the 5′LTR of the packaging component with the cytomegalovirus (CMV) early promoter, replacing the 3′ LTR with SV40 polyA and so on.

In a specific embodiment of the invention, the lentiviral vector is the SV40LT plasmid and the helper packaging plasmid is the pSPAX2, pMD2G plasmid. SV40LT plasmid DNA can transcribe lentiviral genetic material (SV40LTRNA), but cannot translate the outer and protein components of lentiviruses, usually with GFP, resistance genes and reporter genes. psPAX2 is a plasmid capable of expressing a lentiviral coat, and its expression product can cross the cell membrane more easily through the adhesion mechanism. pMD2G plasmid encodes the protein fragment of lentiviral.

The three plasmids were co-transferred into the target cell genome by lipofectamine2000. When the host genome is expressed, the target gene RNA transcribed from the host gene and the protein translated from the psPAX2 and pMD2G genes are assembled into a lentivirus.

The lentiviral vector can efficiently integrate a foreign gene or an exogenous shRNA into the host chromosome, thereby achieving the effect of persistently expressing the sequence of interest.

For some cells that are difficult to transfect, such as primary cells, stem cells, undifferentiated cells, etc., using lentiviral vectors can greatly improve the transduction efficiency of the target gene or target shRNA. The probability of integration of the target gene or the target shRNA into the host cell genome is greatly increased, and the long-term and stable expression of the target gene or the target shRNA can be conveniently and quickly realized.

A lentiviral vector refers to a viral vector derived from human immunodeficiency virus-1 (HIV-1). The lentiviral vector contains the genetic information required for packaging, transfection, and stable integration, which is a major component of the lentiviral vector system. The lentiviral vector carrying the foreign gene is packaged into an infectious virus particle with the help of the lentiviral packaging plasmid and the 293T cell line. Then the foreign gene is expressed in the cell or living tissue by lentiviral infecting the cell or the living tissue.

The immortalized GTKO pig hepatocytes obtained in this application have similar typical morphological characteristics of primary porcine hepatocytes, and biological functions such as ammonia metabolism and urea synthesis. After passage for 40 generations, the expression of hepatocyte-related functional genes and the expression of hepatocyte marker genes showed consistent with the performance of primary GTKO pig hepatocyte cells, and did not exhibit tumorigenicity, and the cell maintains certain proliferation rate.
(1) HepDT has obvious in vitro proliferative activity and proliferates in vitro in diploid. HepDT is cultured in vitro using hepatocyte culture medium with a cell doubling time of 27-32 hours. HepDT has been cultured for over 40 generations, and its proliferative activity has not changed significantly.
(2) The immortalized pig hepatocyte cell line has the following functions as primary hepatocytes: urea synthesis, albumin synthesis.

This application identifies the morphological and biological functions of immortalized GTKO pig hepatocytes, and in some embodiments, includes the following identification items:
(1) HepDT morphological characteristics.
(2) Detection of SV40LT protein expression by immunofluorescence staining.
(3) Detection of Gal antigen gene and its expression by ordinary PCR reaction and Lectin IB4 fluorescent staining.
(4) Determination of glycogen content in HepDT cells by periodic acid-Schiff PAS staining.
(5) Detection of hepatocyte marker gene albumin and hepatocyte nuclear factor 4 (HNF4α) expression by immunofluorescence staining.
(6) RT-PCR was used to detect the expression of hepatocyte function-related genes (cytochrome P4503A, glutamine synthetase (GLUL), glutathione transferase (GST), albumin (Alb), and hepatocyte nuclear factor (HNF4α).
(7) Detection of HNF4α, Alb and SV40LT proteins in HepDT cells by Western Blot.
(8) Determination of urea, alanine aminotransferase and aspartate aminotransferase in culture supernatants of HepDT cells at different time points by biochemical analysis.
(9) Enzyme-linked immunosorbent assay for detection of albumin secretion in culture supernatants of HepDT cells at different culture times.
(10) Cell counting method to plot HepDT cell growth curve

In a third aspect, the invention also provides the use of the immortalized GTKO pig liver cell line obtained by the method of the second aspect for the preparation of an implantable medical device for treating liver disease.

In some embodiments, the implantable medical device is a bioartificial liver (BAL).

In one embodiment, the cell culture mode in the bioartificial liver BAL is obtained by microcarrier culture.

In one embodiment, the cell culture mode in the bioartificial liver BAL is obtained by microencapsulation culture.

In one embodiment, the cell culture mode in the bioartificial liver BAL is obtained by spherical aggregate culture.

In one embodiment, the cell culture mode in the bioartificial liver BAL is obtained by bioreactor culture.

In one embodiment, the cell culture mode in the bioartificial liver BAL is obtained by co-culture.

In a fourth aspect, this invention also provides an application of immortalized GTKO pig hepatocyte cell line in the preparation of a medicament for treating liver failure.

In summary, the advantages of the immortalized GTKO pig hepatocyte cell line and the advantages of the preparation method are as follows:

1. HepDT retains the main features of primary GTKO pig liver cells, such as functional molecular expression of hepatocytes, including urea synthesis and albumin synthesis.

2. HepDT can expanse without restriction in vitro.

3. This invention examines key molecules that mediate rejection in donor xenograft rejection to find important drug targets to intervene. Immortalized GTKO pig hepatocyte cells were used in bioartificial liver treatment to effectively solve the problem of heterogeneous hyperacute immune rejection, which can reduce the use of immunosuppressants, prolong the survival time of transplant recipients and maintain the normal liver function.

4. The immortalized GTKO pig hepatocyte cell line combined with hepatocyte culture technology of this invention can be used for preparing a bioartificial liver support system, preparing a medicament for treating liver failure and used for the treatment of patients with liver failure.

### BRIEF DESCRIPTION OF THE DRAWINGS

FIG. 1 shows an SV40LT Lentiviral Packaging Plasmid Vector.

FIG. 2 shows an h-TERT Lentiviral Packaging Plasmid Vector.

FIG. 3 shows morphological observation results of immortalized GTKO pig hepatocyte cells using ordinary light microscope.

FIG. 4 shows a morphological observation of immortalized hepatocytes HepDT obtained in Example 2 by inverted microscope.

FIG. 5 shows a detection of SV40LT protein expression in HepDT cells by immunofluorescence staining.

FIG. 6 shows a PCR electrophoresis graph of mutant Gal antigen gene in GTKO pig immortalized hepatocyte.

(Lane M stands for: DL2000 Marker (Tiangen Biotechnology (Beijing) Co., Ltd., MD114).

Lane 1,2 represents the wild-type porcine hepatic primary cell Gal antigen gene.

Lane 3,4 represents the Gal antigen gene of GTKO pig immortalized hepatocyte mutation.

FIG. 7 shows a detection of Gal antigen gene and its expression by fluorescent staining of lectin IB4.

FIG. 8 shows an observation of the submicroscopic structure of HepDT cells obtained in Example 2 by 12,000 times under electron microscope.

FIG. 9 shows results of detection of glycogen content in HepDT cells by periodic acid-Schiff PAS staining.

FIGS. 10A-10C show experimental results of detection of hepatocyte marker genes by immunofluorescence staining.

FIG. 10A shows a cellular immunofluorescence staining for detection of hepatocyte marker gene.

FIG. 10B shows a cell immunofluorescence staining detection of hepatocyte marker gene albumin gene Alb.

FIG. 10C shows results of detection of hepatocyte marker gene HNF4a gene by immunofluorescence staining.

FIG. 11 shows an RT-PCR detection of immortalized hepatocyte function-related genes.

FIG. 12 shows a western Blot detection of HNF4α, Alb, SV40LT protein in HepDT cells.

FIG. 13 shows a biochemical analysis of urea, alanine aminotransferase and aspartate aminotransferase in culture supernatants of HepDT cells at different time points.

FIG. 14 shows an enzyme-linked immunosorbent assay for detection of albumin secretion in culture supernatants of HepDT cells obtained in Example 2 at different culture times.

FIG. 15 shows GTKO pig immortalized hepatocyte HepDT and primary hepatocytes were cultured for 1-7 days, respectively, and the growth curve of HepDT cells was obtained by counting method.

### DETAILED DESCRIPTION OF THE EMBODIMENTS

The present invention will be further described below in conjunction with the accompanying drawings and specific embodiments. However, it should be noted that the drawings and the examples are merely illustrative of the invention and are not intended to limit the scope of the invention.

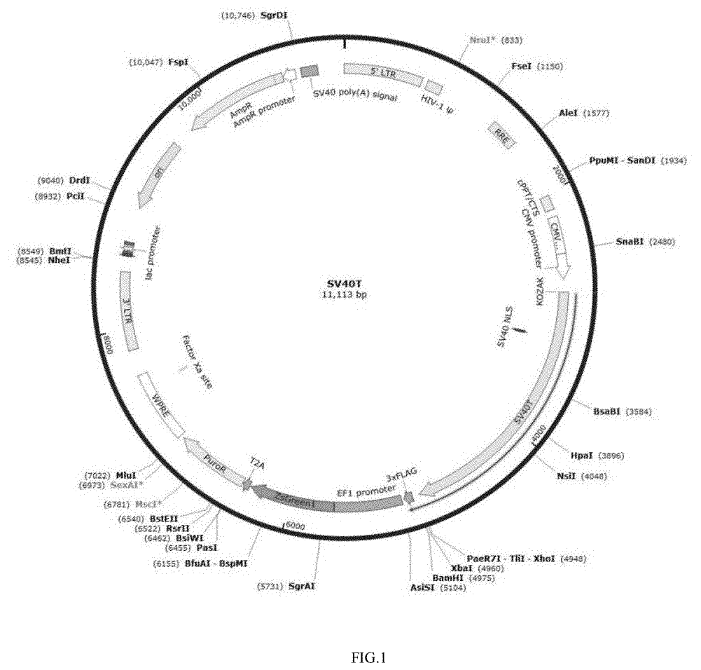

The construction of the recombinant retroviral vector containing the SV40 large T antigen gene was constructed by Hanheng Biotechnology Shanghai Co., Ltd. The SV40LT lentiviral packaging plasmid vector is shown in FIG. 1.

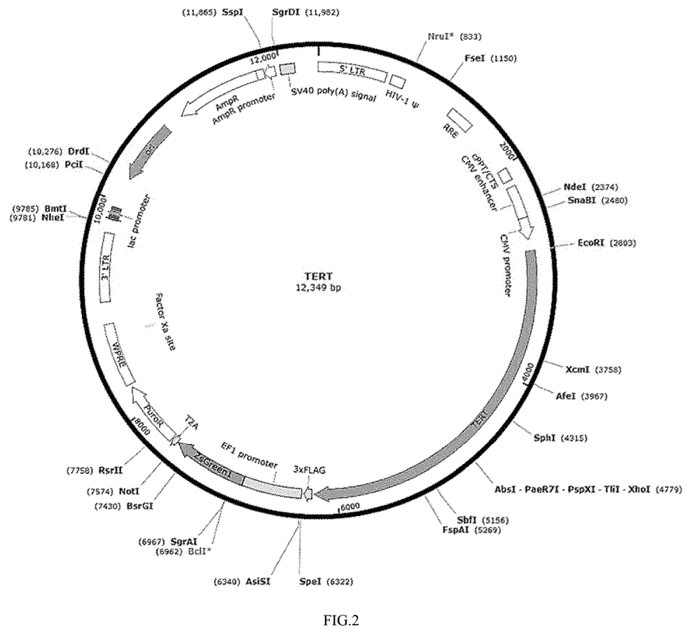

The h-TERT lentiviral packaging plasmid vector is shown in FIG. 2 below.

The h-TERT gene was inserted into the vector MCS region.

SV40LT and hTERT lentivirus is constructed by Han Heng Biotechnology (Shanghai) Co., Ltd.

3 plasmid lentiviral systems: pSPAX2 plasmid (purchased from Addgene, Switzerland), pMD2G plasmid (purchased from Addgene, Switzerland) and lentiviral packaging plasmid (SV40LT lentiviral packaging plasmid vector or h-TERT lentiviral packaging plasmid vector, purchased from Hanheng Biotechnology (Shanghai) Co., Ltd.)

The SV40LT sequence is shown in SEQ ID NO:. 1.

1. SV40LT sequences containing BamH1 and Sal I restriction enzyme cutting site at both ends were synthesized and ligated into pHBLV-CMV vector to obtain pHBLV-CMV-SV40LT.

2. Construction of lentiviral vector encoding the SV40 LT antigen:

The lentiviral vector plasmid pHBLV-CMV was double digested with NEB endonuclease. The conditions of enzyme digestion were 50 μl of total volume adding 1 μl the amount of BamH1 and Sal I in the reaction system, respectively, 37° C. for 1 h, 15 min, then the enzyme was inactivated at 65° C. for 20 min. After that, the ends of the carrier were filled in by adding 1 μl of Klenow enzyme and 2 mM dNTP to the reaction system at room temperature for 15 min, 75° C., 25 min, and finally adding 0.25 μl of cip enzyme for 30 min at 37° C. Finally, the digested product was subjected to 1% agarose gel electrophoresis, and the vector pWPT fragment was recovered by the German Qiagen Gel Recovery Kit (Cat. No. 28704).

The plasmid Plox-Ttag-iresTK and the plasmid Plox-TERT-iresTK were digested with Sal I and EcoRI, and the conditions were 37° C. for 30 min, then the enzyme was inactivated at 65° C. for 20 min. Then, both ends were filled in by adding 1 μl of Klenow enzyme and 2 mM dNTP to the reaction system at the end of the above reaction, leaving it at room temperature for 15 min, then 75° C., 25 min, and finally adding 0.25 μl of cip enzyme for 30 min at 37° C. Finally, the digested products were subjected to 1% low melting point agarose gel electrophoresis, and the SV40T antigen fragment was separately recovered by the Qiagen Gel Recovery Kit (Cat. No. 28704). The sequence alignment of these two fragments confirmed that the SV40 LT antigen fragment was identical to the nt2691-5163 sequence of Genebank No. J02400. Subsequently, the obtained target gene fragment (SV40 T antigen fragment and hTERT fragment) were ligated to the recovered vector fragment, respectively, and the ligation reaction conditions were: using T4 ligase, overnight at 16° C. Thus, a transfection vector plasmid pHBLV-CMV-SV40Tag vector and a pHBLV-CMV-hTERT vector encoding the SV40 T antigen and the hTERT gene, respectively, were obtained.

Example 2, establishment of immortalized GTKO pig liver cell line (HepDT):

Host material: α-1,3-galactosyltransferase pig was provided by Prof. Pan Dengke from the Beijing Academy of Agricultural Sciences.

1. Packaging and titration of lentiviral particles.

2. Plasmid packaging system: pSPAX2 (purchased from Addgene, Switzerland), pMD2G plasmid (purchased from Addgene, Switzerland) and lentiviral packaging plasmid (SV40LT lentiviral packaging plasmid vector or h-TERT lentiviral packaging plasmid vector (purchased from Hanheng Biotechnology) (Shanghai Co., Ltd).

The mass ratio of the three plasmids was 1 μg of pHBLV-CMV vector carrying SV40LT, 750 ng psPAX2 packaging plasmid, 250 ng pMD2.G envelope plasmid.
(1) 293T cells were transfected with recombinant lentiviral vector pHBLV-CMV encoding SV40 LT antigen. Then 293T cells were cultured in DMEM containing 10% FCS and 100 ug/ml cyan/streptomycin in DMEM. After that 293T cells were seeded at 3×10⁶ cells/dish in 100 mm petri dishes 24 h prior to transfection. Finally fresh medium was replaced 2 h before transfection.
   Each dish was transfected with 20 ug of plasmid DNA, including: 10 ug transfection vector plasmid (pWPT-GFP or pWPT-SV40Tag or pWPT-hTERT), 3.5 ug capsid coding plasmid and 6.5 ug packaging plasmid. Resuspend this 20 ug plasmid vector in a volume of 450 μl with 0.1×TE solution (1 mM Tris-HCl and 0.1 mM EDTA), then add 50 μl of 2.5 M CaCl 2 solution and mix gently. Then, 500 μl of 2× HEPES buffered saline (0.1 M HEPES, 0.281 M NaCl, and 1.5 mM Na2HPO4 [pH 7.12]) were added dropwise while vortexing. The mixture was placed at room temperature for 30 min, then added to the cultured cells, and the suspension was slowly added. The medium in the dish was gently shaken while being added. Then the culture dish is placed in a 37° C. incubator, replacing 10 ml fresh medium after 16 hours. The medium was collected and replaced every 24 hours thereafter, and the collected supernatant was centrifuged at 900 g for 10 min to remove cell debris and filtered through a 0.2-μm pore size filter. The lentiviral particles carrying the GFP gene and the SV40 T antigen, which are infectious and replication-defective, were separately packaged by the above methods, and then directly used or frozen at −80° C. for use.
(2) Titration of virus particle titer
   HeLa cells were seeded in a 12-well plate at a density of 1×105 cells/well and cultured in DMEM medium supplemented with 10% FCS, 2 mM glutamine, and 10 mM Hepes (GibcoBRL, Life Technologies). The cells were cultured overnight at 37° C. in a 5% CO₂ incubator (American Thermo company, teri-Cycle).
   Continuously diluted lentiviral particles and a final concentration of 8 μl/ml polybrene (Jiman Biotechnology (Shanghai) Co., Ltd., item number GM-040901) were used to continue to culture Hela cells for 48 h. The cells were collected by trypsinization, centrifuged, and the supernatant was discarded. The pellet was resuspended in 300 μl of 3.7% formaldehyde/PBS, and the ratio of EGFP-positive cells was analyzed by FACS. Titer is expressed as transducing unit/ml (TU/ml). The virus particle titer measured in this experiment was 108 TU/ml.

2. Acquisition of primary isolated GTKO pig hepatocytes

GTKO pig primary hepatocytes were extracted from GTKO pigs using a two-step method of collagenase/DMEM combined with a stable peristaltic pump perfusion system.
(1) Selecting GTKO pigs born about 20 days old (provided by Mr. Pan Dengke from Beijing Academy of Agricultural Sciences). Pig hungry one day before the experiment. Intramuscular anesthetic Shutai for anesthesia. In the sterile animal operation room, using the surgical blade to open the abdominal cavity of the pig, finding the hepatic portal vein and the inferior vena cava, and performing preliminary dissociation to facilitate intubation.
(2) The catheter was placed in the hepatic portal vein, aseptically ligated and fixed with a vascular clamp, and the inferior vena cava was cut open and perfused with saline to the inferior vena cava.
(3) Anatomically removing the GTKO pig liver and connecting it through a catheter to the assembled perfusion peristaltic pump system (Baoding Lange Constant Flow Pump Co., Ltd., BT100-2J/YZ1515x). Repeated perfusion with 37% 0.5% collagenase/DMEM until the liver tissue is soft.
(4) Placing liver tissue on ice pack to stop digestion, using the tweezers to tear the liver capsule, and adding DMEM medium to wash the liver cells digested from the liver tissue. Discarding the remaining undigested liver tissue blocks and filtering the cells with 100 mesh and 200 mesh screens.
(5) Collecting filtrate containing hepatocytes and centrifuge at 1000 rpm for 3 minutes.
(6) Discarding the supernatant, adding the red blood cell lysate to suspend the liver cells, and the red blood cells were lysed for 3 min. Then adding DMEM medium to suspense cells, and centrifuge at 50 g for 3 min.
(7) Discarding the supernatant, the cells were suspended in DMEM medium, and centrifuged at 10 g for 3 min.
(8) Finally, the cells were suspended in 15% FBS HM medium, and were seeded in a culture flask.
(9) After 2-3 hours, the cells adhered to the plate, changing fluid to remove unattached cells and continue to culture.

Primary isolated GTKO pig liver cells were seeded in 24-well plates and suspended in DMEM/F-12 medium (Thermo, 11320082). The obtained primary hepatocytes have high viability.

3. Screening of recombinant GTKO pig hepatocyte clones transfected with lentiviral particles with green fluorescent protein (GFP gene, SV40 T antigen)

Freshly isolated GTKO pig primary hepatocytes were cultured with 10 mg/L HGF, 10 ug/L EGF, 250 IU/L insulin, 200 ug/L Dexamethasone, 2 mmol/L glutamine, 10% fetal bovine serum culture medium (Hepatocyte Medium (ScienCell, USA, USA) at a concentration of 8.0×10⁵ in cell T25 plastic flask (ThermoFisher, Cat. No. 136196) placed in a 37° C., 5% CO2 incubator.

After 24 hours, the culture medium was changed, and the freshly cultured primary GTKO pig liver cells were infected with the supernatant of the recombinant retrovirus containing SV40 large T antigen (polybrene concentration was 8 ug/ml). 1 week later, drug pressure screening was performed with 500 ug/ml of G418 (supplier Thermo Fisher, model 10131027) for 4 weeks. When the cell clone is grown to a diameter of 1.0-2.0 cm, the picked cell clone is inoculated into a 6-well plate and cultured to obtain an immortalized GTKO pig liver cell line HepDT.

This study used α-1,3-galactosyltransferase knockout pig cultivated by prof. Pan Dengke of the Beijing Academy of Agricultural Sciences as animal material. The immortalized GTKO pigs hepatocyte cell line was obtained and identified. Inserting a gene sequence (SEQ ID NO: 1) into the gene of wild-type α-1,3-galactosyltransferase (GGTA1) to silence the gene to produce GTKO pig.

1. Morphological observation of ordinary light microscope.

The highly viable immortalized GTKO pig hepatic cell line cultured for 24 hours obtained in Example 2 was observed with OLYMPUS inverted fluorescence microscope (Olympus, Japan, Model IX71FL+DP72) according to the procedure shown in the instruction manual.

As shown in FIG. 3, graph A shows freshly distributed porcine hepatocyte with a single distribution, the shape is round, elliptical, the cell outline is clear, the cell membrane is intact, the cytoplasm and nucleus are evenly distributed, and are clearly visible, generally mononuclear.

Graph B shows a close connection of cells after culture and adherence growth. Most of the pig liver cells are binuclear cells with a flat shape and a polygonal shape.

C. Multiple cells are arranged in clusters and clusters, and more binuclear cells are visible at the same time.

2. SV40LT lentivirus titer calculation

Virus titers were determined according to dilution notation. (https://wenku.baidu.com/view/fd2a91e54a7302768f9939c0.html)

SV40LT lentiviral titer detection by inverted fluorescence microscopy (Shanghai Biem Optical Instrument Co., Ltd., Item No. BM-38X) according to virus titer dilution counting method.

The 293T cells infected with the virus were observed under a fluorescence microscope, and the percentage of fluorescence in the 10 to 30% of the wells was calculated according to the following formula:

Titer (TU/mL)=cell number×fluorescence percentage×MOI(1)×virus dilution factor×103 Calculate SV40LT lentivirus titer, this SV40LT titer=4*10\hat{ }4*20%*MOI(1)* virus dilution factor (30)*103=2*108 TU/mL

3. The fluorescence after SV40LT lentivirus infection of GTKO pig primary liver cells for 72 hours.

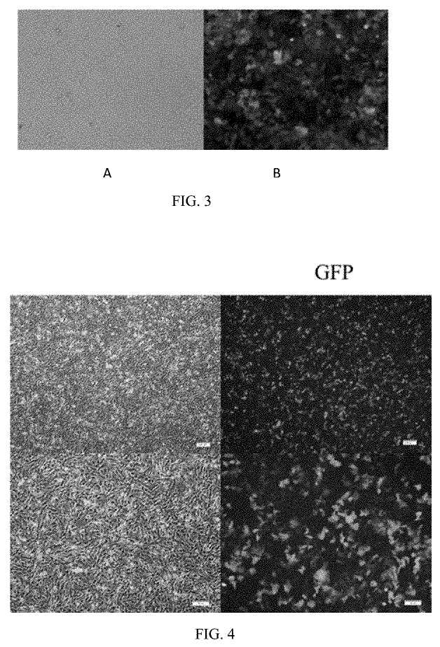

The SV40LT lentivirus-infected GTKO pig primary hepatocytes obtained in Example 2 were observed by fluorescence microscopy, and the results are shown in FIG. 4:
A GTKO pig primary hepatocyte morphology after infection with SV40LT lentivirus under a 4× microscope.
B. GFP fluorescence of GTKO pig primary hepatocytes after infection with SV40LT lentivirus under a 4× microscope.
C GTKO pig primary hepatocyte morphology after infection with SV40LT lentivirus under a 10× microscope.
D. GFP fluorescence of GTKO pig primary hepatocytes after infection with SV40LT lentivirus under 10× microscopic.

According to the GFP fluorescent label carried by the virus, the efficiency of the original GTKO pig liver cells infected with SV40LT lentivirus was about 60%.

4. GTKO primary hepatocytes were infected with lentivirus and visualized by fluorescence staining after one-week puromycin selection.

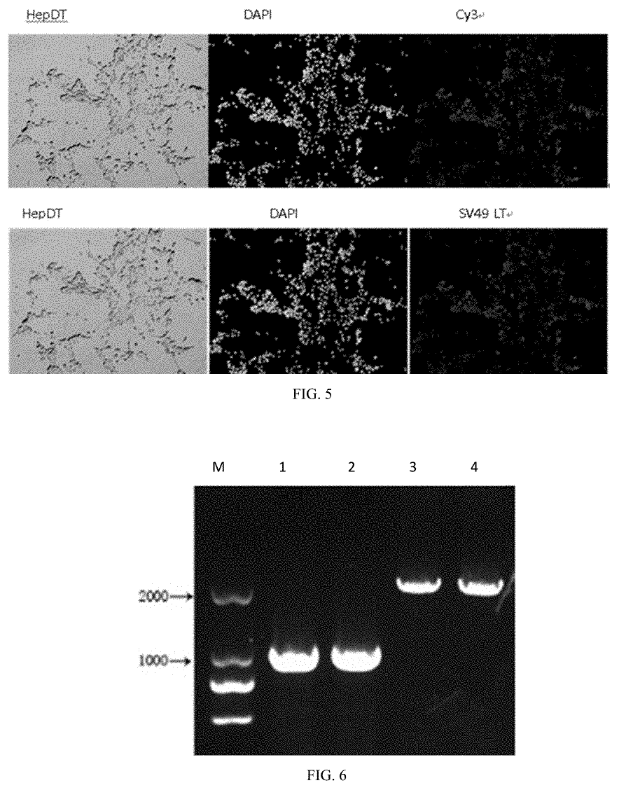

According to the OLYMPUS inverted fluorescence microscope instructions, fluorescence staining of GTKO primary hepatocytes infected with lentivirus after one-week puromycin selection was performed by inverted fluorescence microscope (manufactured by Japan OLYMPUS, Cat. No. IX71FL+DP72). The results are shown in FIG. 5.
A is a 10-fold microscopic morphology of hepatocyte monoclonal cell pellets after one week of puromycin screening.
B is under a 10-fold microscopically GTKO pig hepatocyte monoclonal cell cluster with GFP fluorescence after one week of puromycin screening.

After 10 weeks of puromycin screening, GTKO pig hepatocyte monoclonal cell clusters with GFP fluorescence showed successful infection with SV40LT lentivirus. And the SV40LT antigen is expressed in the cell to allow the primary cells to proliferate.

5. Morphological observation of GTKO immortalized hepatocytes HepDT

Morphological observation of the GTKO immortalized hepatocyte HepDT obtained in the third step of Example 2 was carried out by an inverted fluorescence microscope (manufactured by Olympus, Japan, Cat. No. IX71FL+DP72) according to the OLYMPUS inverted fluorescence microscope instruction. The results shown in FIG. 6 indicate that immortalized pig liver cells are regular in shape, uniform in size, and triangular, all of which are monocytes.
A immortalized liver cell morphology under 4 times microscope
B immortalized hepatocyte GFP fluorescence under 4 times microscope
C immortalized liver cell morphology under 10 times microscope
D immortalized hepatocyte GFP fluorescence under 10 times microscope

The results showed that the immortalized pig liver cells were regular in shape and uniform in size and triangular in shape, all of which were monocytes.

6. Detection of SV40LT protein expression in immortalized GTKO pig liver cells by immunofluorescence staining.

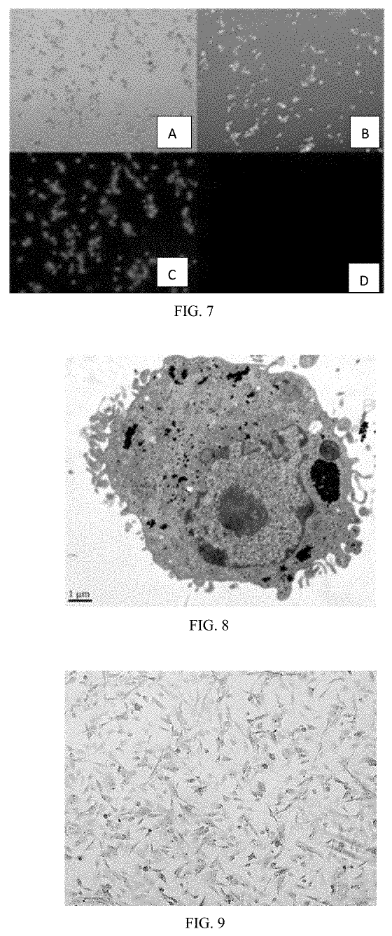

Inoculate the immortalized hepatocytes obtained in step 3 of Example 2 by the method of cell slide (manufactured by Solarbio, model YA0350) according to the procedure shown below. Cell immunofluorescence staining was performed according to the procedure shown below, and its expression in the nucleus was detected by SV40LT antibody. The results are shown in FIG. 7.

Hepatocytes growing on glass coverslips:
1) After digesting the cells with trypsin, resuspend the cells in complete medium, mix thoroughly by pipetting, and make a single cell suspension, and count.
2) Place the sterile slides in a 24-well plate and add the cell suspension to the 24-well plate at 2×10 5 cells per well.

Cellular immunofluorescence staining steps:
1) Wash the cells twice with PBS for 3 minutes each time.
2) Fixation: Fix the cells in 4% paraformaldehyde (Biyuntian Biotechnology Co., Ltd., P0099) for 15 minutes.
3) Remove paraformaldehyde, wash the cells 3 times with PBS for 3 minutes each time.
4) Permeabilization: 0.3% Triton X-100 in PBS, permeabilized cells for 20 minutes at room temperature.
5) Remove Triton X-100 (purchased from sigma, item number T9284), wash the cells 3 times with PBS for 3 minutes each time.
6) Blocking: Block with 1% serum homologous to the secondary antibody for 1 hour.
7) Primary antibody: add enough appropriate concentration of primary antibody and incubate overnight at 4° C.
8) Remove the primary antibody and wash the cells 3 times with PBS for 3 minutes each time.
9) Secondary antibody: add enough amount of secondary antibody at appropriate concentration, incubate at room temperature for 1 hour in the dark.
10) Remove the secondary antibody and wash the cells 3 times with PBS for 3 minutes each time.
11) Nuclear staining: 0.5 μ0/ml DAPI incubation in the dark for 5 min.
12) Lightly wash the cells with PBS 3 times for 5 minutes each time to wash off the excess DAPI (Beijing Reagan Biotechnology Co., Ltd., Item No. 1108A17).
13) Remove the cell slide from the tweezers, seal with a sealing solution containing anti-fluorescence quencher, and observe and collect the image under a fluorescence microscope.

The red fluorescent SV40LT antigen was observed to be identical to the nuclear localization DAPI, indicating that the SV40 large T antigen was successfully expressed in the nucleus of immortalized hepatocytes.
A shows HepDT morphology after immunofluorescence staining at 4 times microscope.
B shows HepDT 4′,6-diamidino-2-phenylindole (DAPI) immunofluorescence staining, the nucleus is under 4 times microscope.
C shows HepDT SV40LT expression in immunofluorescence staining under 4 times microscope (primary antibody is SV40LT monoclonal antibody, secondary antibody is Cy3 labeled antibody)

DAPI, 4′,6-diamidino-2-phenylindole, is a fluorescent dye that binds strongly to DNA and is commonly used for fluorescence microscopy. Because DAPI can penetrate intact cell membranes, it can be used for staining of living cells and fixed cells.

Cy3, the cyanine dye is fluorescently labeled and is often used for biomolecular labeling, fluorescence imaging and other fluorescent bioanalysis.

DAPI was purchased from Beijing Reagan Biotechnology Co., Ltd., and Cy3 fluorescent labeled secondary antibody was purchased from Kangwei Century Biotechnology Co., Ltd.

SV40LT monoclonal antibody was purchased from Abcam company, article number: ab16879.

Immunofluorescence staining was used to detect the expression of SV40LT antigen in cells by Cy3-labeled antibody. The results showed that SV40LT antigen was expressed in GTKO pig immortalized hepatocytes, and the expression position was located in the nucleus. SV40LT protein can play a role in promoting cell proliferation in the nucleus.

7. The PCR reaction detects the Gal antigen gene and its expression.

The forward primer of the Gal antigen gene detection is 5′-3′: GGATGCTTCCTCTAGTCTGTGATG, as shown in SEQ ID NO: 2

The reverse primer 5′-3′: CTCTAGCCTACCCAGAACTGCAGAG, as shown in SEQ ID NO: 3

| | | | | | |
|---|---|---|---|---|---|
| Reaction | 2xPrimix Taq | 12.5 | μl | | |
| system | forward primer | 1 | μl | | |
| | reverse primer | 1 | μl | | |
| | template | 100 | ng | | |
| | sterile water | add water to 25 | μl | | |
| Reaction | 95° C. | 5 | min | | |
| procedures | 95° C. | 30 | s | | |
| | 60° C. | 30 | s | {close oversize brace} | 35 cycles |
| | 72° C. | 2 | min | | |
| | 72° C. | 10 | min | | |

The reaction product was stored at 4° C. The PCR results are shown in FIG. 8.
Lane M stands for: DL2000 Marker (Tiangen Biotechnology (Beijing) Co., Ltd., MD114).
Lane 1,2 represents the Gal antigen gene of wild-type porcine hepatic primary cell.
Lane 3,4 represents the mutant Gal antigen gene of GTKO pig immortalized hepatocyte.

PCR results showed that GTKO pig immortalized hepatocytes successfully expressed mutant Gal antigen gene.

8. Detection of Gal antigen gene and its expression by fluorescent staining of plant lectin IB4.

Lectin IB4 was purchased from VECTOR LABORATORIES NO DL-1207

As shown in FIG. 9.
A Cell morphology of wild pig hepatic primary cell after lectin B4 fluorescent staining.
B Cell morphology of GTKO pig hepatic primary cell after lectin B4 fluorescent staining.
C Wild pig hepatic primary cell lectin B4 fluorescent staining.
D GTKO pig hepatic primary cell lectin B4 fluorescent staining.

The results showed that GTKO pig immortalized hepatocytes did not express α-1,3-galactose.

9. Electron microscopic observation of HepDT cells submicroscopic structural features under 120,000 times.

Steps to observe HepDT cells under electron microscope:
1) After washing the cells in the culture plate with 0.1 M PBS, the cells were fixed by adding 2.5% glutaraldehyde for 1 hour.
2) Wash the cells 3 times with 0.1 M PBS for 5 min each time.
3) 1% citrate was used to fix cells for 1 h, then the cells were washed 3 times with 0.1 M PBS for 5 min each time.
4) 4% aqueous uranyl acetate solution staining for 30 min.
5) 50%, 70%, 90% alcohol dehydrated in turn, each 15 min.
6) 100% alcohol dehydration for 20 min.
7) 100% acetone dehydration for 20 min.
8) Anhydrous acetone and embedding agent mixed in a 1:1 volume to penetrate the tissue and oscillate for 2 h.
9) Pure embedding agent penetrates the tissue and oscillates for 2 h.
10) The pure embedding agent is embedded and placed in an oven for polymerization, followed by 37° C., 24 hours; 45° C., 24 hours; 60° C., 48 hours.
11) Repair and ultra-thin section.
12) 4% uranyl acetate staining for 20 min, bismuth lead staining for 5 min.
13) TECNAI 10 transmission electron microscope observation.

Chemical reagents were purchased from Tianjin Tianli Chemical Reagent Co., Ltd. Transmission electron microscope (Japan JEOL, model JEOL-100CXII).

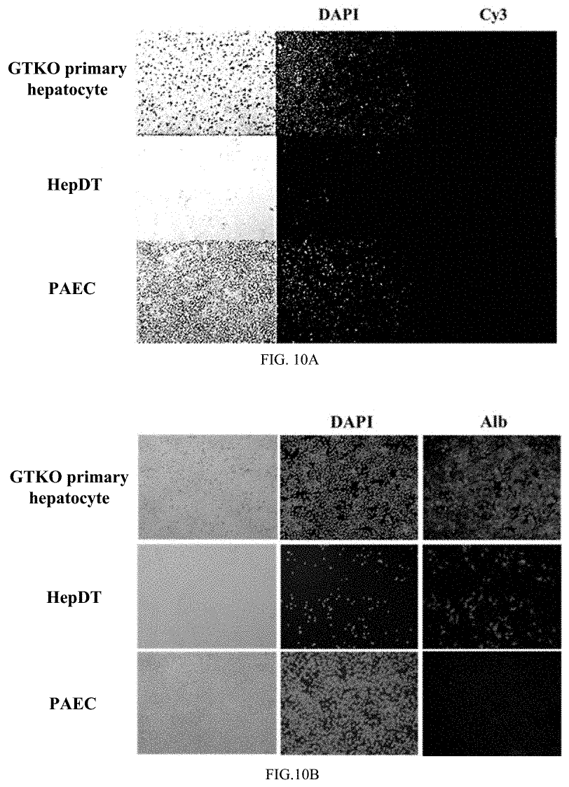

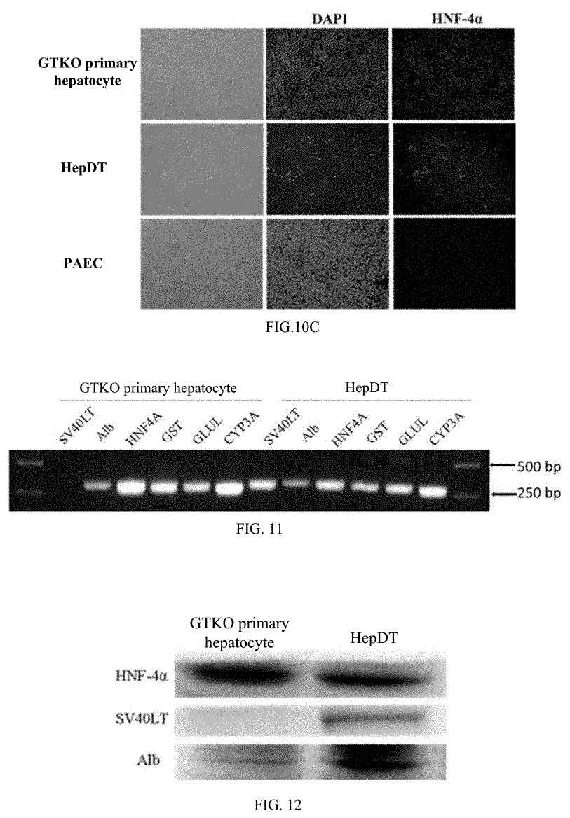

The results are shown in FIG. 10. Under the electron microscope, hepatocytes HepDT showed large nucleoli, microvilli on the cell membrane, abundant cytoplasmic glycogen particles, mitochondria, endoplasmic reticulum and organelles.

10. Determination of glycogen content in HepDT cells by periodic acid-Schiff PAS staining

Periodic acid-Schiff stain, the principle: periodic Acid-Schiff stain, which uses periodic acid to oxidize the intracellular polysaccharides to produce free aldehyde groups and acid groups, and then reacts with Schiff's dye to form purple-red compounds in the cytoplasm, is a method for the glycogen storage detection. This staining method was used to initially identify hepatocytes.

The dyeing procedure refers to Solarbio Bios glycogen PAS staining solution (Cat. No. G1360). As shown in FIG. 11, after staining with glycogen, the cells showed purple-red glycogen particles in the cytoplasm, indicating that the cells have glycogen synthesis ability.

11. Cellular immunofluorescence staining for detection of hepatocyte marker gene albumin (Alb) expression.

The procedure and the source of the reagents used, the type of microscope used for the observation, or the experimental procedure are as described in 6 above. The results are shown in FIG. 12A, FIG. 12B, and FIG. 12C.

Cellular immunofluorescence results showed that Alb was expressed in both GTKO pig primary hepatocytes and immortalized hepatocytes, while negative control pig endothelial cells did not express Alb in PAEC.

12. RT-PCR detection of hepatocyte function-related genes

Cytochrome CYP3A (P450 3A), glutamine synthetase GLUL, glutathione transferase GST, albumin Alb, and hepatocyte nuclear factor HNF4 are important molecules for hepatocytes to exert metabolism and function.

| | | Product |
|---|---|---|
| gene | Primer 5′-3′ | length |
| HNF4A | Forward: TCAGAAGGTGCCAACCTCAA, as | 307 |
| | shown in SEQ ID NO: 4; | |
| | Reverse: CGTAGCTTGACCTGCGAGTG, as | |
| | shown in SEQ ID NO: 5. | |
| GLUL | Forward: CCATGCGAGAGGAGAATGGT, as | 294 |
| | shown in SEQ ID NO: 6; | |
| | Reverse: TGCGGATGAGAGCTTCTGTC, as | |
| | shown in SEQ ID NO: 7. | |
| GSTA1 | Forward: CCGAGGCAGAATGGAGTGTA, as | 298 |
| | shown in SEQ ID NO: 8; | |
| | Reverse: CAGTGGCAACAGCAAGATCA, as | |
| | shown in SEQ ID NO: 9. | |
| CYP3A29 | Forward: GGCCAAGACCTCTGCCTTATT, as | 289 |
| | shown in SEQ ID NO: 10; | |
| | Reverse: GTCGGAGACAGCAATGTTCG, as | |
| | shown in SEQ ID NO: 11. | |
| SV40LT | Forward: ATTGCCTGGAACGCAGTGAG, as | 310 |
| | shown in SEQ ID NO: 12; | |
| | Reverse: CCTGAGTCTTCCATGTTCTTCTCC, | |
| | as shown in SEQ ID NO: 13. | |
| Alb | Forward: GCCTCTTGTGGATGAGCCTAA, as | 311 |
| | shown in SEQ ID NO: 14; | |
| | Reverse: CCAAGGACTCTGTGCAGCAT, as | |
| | shown in SEQ ID NO: 15. | |

| | | | | | |
|---|---|---|---|---|---|
| Reaction | 2xPrimix Taq | 12.5 | μl | | |
| system | Forward primer | 1 | μl | | |
| | Reverse primer | 1 | μl | | |
| | template | 100 | ng | | |
| | sterile water | add water to 25 | μl | | |
| Reaction | 95° C. | 5 | min | | |
| procedure | 95° C. | 30 | s | | |
| | 60° C. | 30 | s | {close oversize brace} | 35 cycles |
| | 72° C. | 1 | min | | |
| | 72° C. | 10 | min | | |
| | 4° C. | | | | |

PCR instrument (Eppendorf, Germany, model Eppendorf AG 22331 Hamburg), total RNA inversion kit (Dalian Bao Bioengineering Co., Ltd., 6110A), PCR Premix Taq enzyme (Dalian Bao Bioengineering Co., Ltd., RR902Q)

As shown in FIG. 13, RT-PCR results showed that, like primary hepatocytes, GTKO immortalized hepatocytes express hepatocyte marker genes and their metabolic function-related genes Alb, HNF-4α, GST, GLUL, CYP3A. The SV40LT gene is only expressed in immortalized hepatocytes.

The expression of these molecules in immortalized hepatocytes by RT-PCR indicates that immortalized hepatocytes have the biological function of normal hepatocytes.

13. Western Blot was used to detect the expression of HNF4α, Alb and SV40LT proteins in HepDT cells:

The brief experimental steps of Western Blot are as follows:
1) Wash the liver cells in the culture plate twice with cold PBS, add RIPA lysate containing 1 mM PMSF (Beyotime Biotechnology Co., Ltd., P0013B), and lyse the cells on ice for 30 min.
2) Collect cells by cell scraping, centrifuge at 14000 rpm for 15 min at 4° C., and collect the supernatant.
3) BCA method was used for protein quantification, then denature the protein at 100° C. for 10 min.
4) Separating the protein by electrophoresis on a 10% SDS-PAGE, and transferring the protein to a polyvinylidene fluoride (PVDF) membrane (millipore, IPVH00005, USA).
5) Incubate the PVDF membrane with 5% skim milk TBST (TBS supplemented with 0.1% Tween 20) for 1 hour at room temperature, then incubate the membrane with primary antibody at 4° C. overnight.
6) The membrane was washed with TBST, then incubated with the secondary antibody for 1 hour at room temperature, and then washed three times with TB ST. Finally, the Western blot was visualized by an enhanced chemiluminescence detection reagent and analyzed using ImageJ software.

The experimental procedure refers to the first edition of the Fourth Military Medical University Press, December 2011, “Practical Experimental Technology of Molecular Biology”.

The instruments used were: chemiluminescence system (US BIO-RAD, model ChemiDoctm XRS+), electrophoresis system (US BIO-RAD, model POWERPAC BASIC), anti-SV40T primary antibody (American abcam, ab16879), Albumin monoclonal antibody (Proteintech, Item No. 66051-1-1-Ig), anti-HNF-4 alpha monoclonal antibody (American abcam, ab41898).

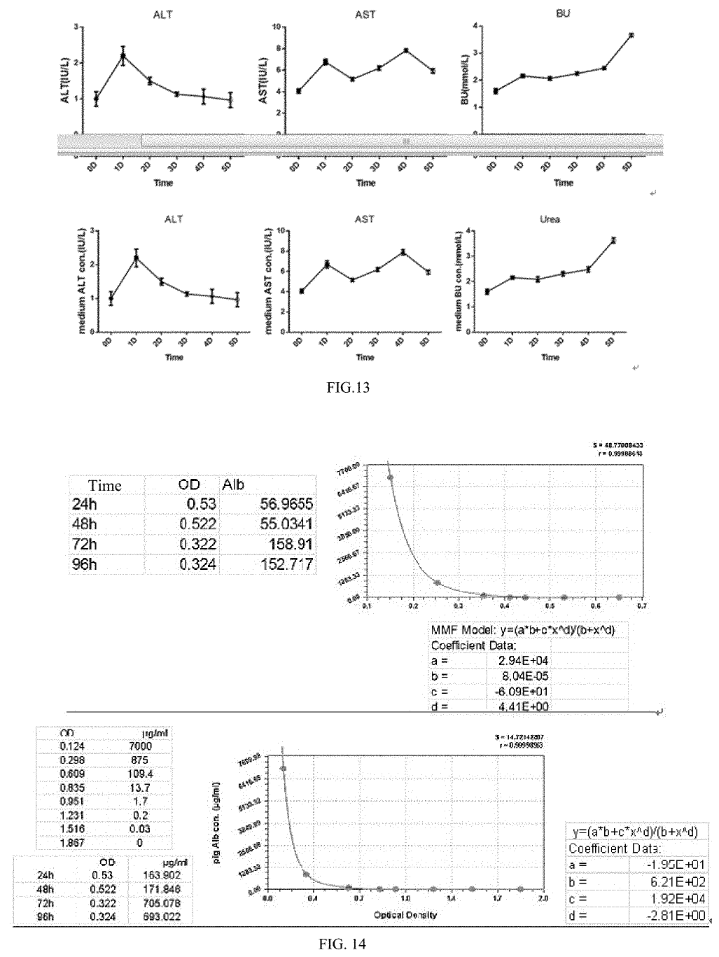

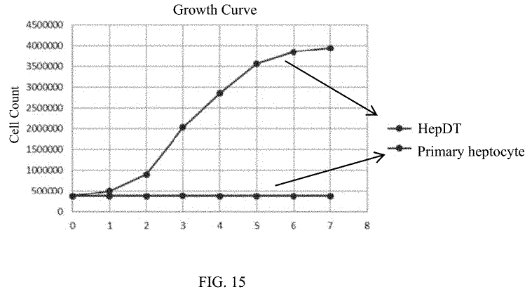

The Western blot results shown in FIG. 14 showed that Alb, HNF-4α protein were simultaneously expressed in GTKO pig primary hepatocytes and the immortalized hepatocytes, while SV40LT protein only expressed in immortalized hepatocytes.

14. Biochemical analysis was used to detect the content of urea, alanine aminotransferase and aspartate aminotransferase in culture supernatant of HepDT cells at different time points:

Automatic biochemical analyzer (USA RAYTO, model Chemray 240). The kit was purchased from Zhongsheng Beikong Biotechnology Co., Ltd.: albumin assay kit (bromocresol green method), aspartate aminotransferase assay kit (aspartate substrate method), alanine Aminotransferase assay kit (alanine substrate method) and urea assay kit (urease-glutamate dehydrogenase method).

AST, ALT, Urea detection operations are performed according to the kit instructions.

As shown in FIG. 15, the results showed that urea, alanine aminotransferase, and aspartate aminotransferase were detected in the immortalized hepatocyte culture supernatant. The concentration of ALT and AST in the cell supernatant is always at a low level, which indicates that the cell membrane integrity is good during the growth process. The concentration of Urea in the supernatant of the cells showed a gradually increasing trend, indicating that the cells have ammonia metabolism ability.

15. Enzyme-linked immunosorbent assay Elisa was used to detect albumin secretion (μg/ml) in cell culture supernatants at different culture times:

After GTKO pig immortalized hepatocytes were cultured for 24 h, 48 h, 72 h, 96 h, the cell culture supernatant was collected. ELISA was used to detect the content of Alb in the supernatant.

Enzyme-linked immunosorbent assay using Pig Albumin Elisa assay kit (Wuhan Huamei Bioengineering and Co., Ltd., CSB-E16207p). The operation steps are as follows:
1) Place the reagents at room temperature for at least 30 minutes, and prepare the reagents required according to the kit instructions.
2) Loading: Set blank holes, standard holes, and test sample holes. Blank wells were not added to any solution. Add 50 μl of standard or test sample to each well. 50 μl of the antibody working solution was immediately added, and the blank well was not added. Gently shake and mix, cover the plate, and incubate at 37° C. for 60 minutes.
3) Discard the liquid in the well, dry and wash the plate 3 times. Soak for 2 minutes each time, 200 μl per well, dry.
4) 100 μl of enzyme-binding working solution was added to each well, and blank wells were not added. Gently shake and mix, cover the plate, and incubate at 37° C. for 60 minutes.
5) Discard the liquid in the well, dry and wash the plate 5 times. Soak for 2 minutes each time, 200 μl per well, dry.
6) 90 μl of the substrate solution was added to each well in turn, and developed at 37° C. for 20 minutes in the dark.
7) 50 μl of the stop solution was added to each well in turn to terminate the reaction.
8) The optical density (OD value) of each well was sequentially measured by a microplate reader at a wavelength of 450 nm within 5 minutes after the termination of the reaction.

The microplate reader uses an ultra-microplate spectrophotometer (Biotek, Epoch, USA).

Using Curve Expert1.4 software to draw the ELISA standard curve, inputting the standard albumin concentration and OD value, the software automatically generates the standard curve and equation, input the average OD value of the sample to be tested, and calculate the corresponding albumin concentration of each sample.

The curve shown in FIG. 16 is an Elisa standard curve prepared according to different concentration standard solutions, and the average concentration of Alb is obtained by the OD value tested after three tests.

16. GTKO pig immortalized hepatocyte HepDT and primary hepatocytes were cultured for 1-7 days respectively, and the growth curve of HepDT cells was drawn by cell counting.

The HepDT cell growth curve is shown in FIG. 17, which shows that the immortalized GTKO pig hepatocyte HepDT conforms to the “S” growth characteristics.

The above test results indicate that the immortalized GTKO pig liver cell HepDT obtained by this invention can synthesize various functional molecules required for liver function. The immortalized GTKO pig liver cells obtained by the invention have important application prospects in the field of liver disease and liver transplantation therapy.

What is claimed is:

1. An immortalized α-1,3-galactosyltransferase gene knockout (GTKO) pig hepatocyte cell line, deposited at the China General Microbiological Culture Collection Center on Apr. 25, 2018, with the accession number of CGMCC No.15590.

2. The immortalized GTKO pig hepatocyte cell line according to claim 1, having the following biological characteristics:
(1) the immortalized GTKO pig hepatocyte cell line have proliferative activity and proliferates in a diploid manner in vitro;
(2) the immortalized GTKO pig hepatocyte cell line does not express α-1,3-galactosyltransferase;
(3) the immortalized GTKO pig hepatocyte cell line has abilities of bilirubin metabolism, urea synthesis, and albumin synthesis of primary pig hepatocytes.

3. A method for preparing the immortalized GTKO pig hepatocyte cell line according to claim 1, comprising the following steps:
transfecting normal GTKO pig primary hepatocyte cells freshly extracted with a recombinant lentiviral vector containing an SV40T antigen gene to obtain transfected cells, and then
performing monoclonal screening on the transfected cells to obtain the immortalized GTKO pig hepatocyte cell line.

4. The method for preparing the immortalized GTKO pig hepatocyte cell line according to claim 3, wherein, the SV40T antigen gene carried by the recombinant lentiviral vector is transfected into the normal GTKO pig primary hepatocyte cells, and the recombinant lentiviral vector carrying the SV40T antigen geneis pWPT-SV40Tag.

5. The method for preparing immortalized GTKO pig hepatocyte cell line according to claim 4, wherein, the recombinant lentiviral vectors carrying the SV40T antigen gene is transfected into the normal GTKO pig primary hepatocyte cells by the following steps:
inserting the SV40T antigen gene into a multiple cloning site of a lentiviral vector pWPT to construct the recombinant lentiviral vector carrying the SV40T antigen gene; then
packaging the recombinant lentiviral vector carrying the SV40T antigen gene into a lentiviral particle, wherein the lentiviral particle is infectious but has replication defects; and
using the lentiviral particle to infect the normal GTKO pig primary hepatocyte cells.

6. The method for preparing the immortalized GTKO pig hepatocyte cell line according to claims 3, wherein, the normal GTKO pig primary hepatocyte cells are α-1,3-galactosyltransferase gene knockout pig adult hepatocytes.

7. The method for preparing the immortalized GTKO pig hepatocyte cell line according to claim 3, wherein, after monoclonal screening to obtain the immortalized GTKO pig hepatocyte cell line, the immortalized GTKO pig hepatocyte cell line is expanded on microcarriers in vitro.

8. The method for preparing the immortalized GTKO pig hepatocyte cell line according to claim 7, wherein, the immortalized GTKO pig hepatocyte cell line and the microcarriers are suspended in a hepatocyte medium for an expansion culture, wherein the microcarriers are at 50,000-900,000 cells/ml in the expansion culture.

9. A method of using the immortalized GTKO pig hepatocyte cell line according to claim 1 in preparing a bioartificial liver, comprising the following step: using the immortalized GTKO pig hepatocyte cell line to prepare the bioartificial liver.

10. A method of using the immortalized GTKO pig hepatocyte cell line according to claim 1 in preparing a medicine for treating a liver failure, comprising the following step: using the immortalized GTKO pig hepatocyte cell line to prepare the medicine.

11. The method for preparing the immortalized GTKO pig hepatocyte cell line according to claims 4, wherein, the normal GTKO pig primary hepatocyte cells are α-1,3-galactosyltransferase gene knockout pig adult hepatocytes.

12. The method for preparing the immortalized GTKO pig hepatocyte cell line according to claims 5, wherein, the normal GTKO pig primary hepatocyte cells are α-1,3-galactosyltransferase gene knockout pig adult hepatocytes.

13. The method for preparing the immortalized GTKO pig hepatocyte cell line according to claim 4, wherein, after monoclonal screening to obtain the immortalized GTKO pig hepatocyte cell line, the immortalized GTKO pig hepatocyte cell line is expanded on microcarriers in vitro.
